(19) United States
(12) Patent Application Publication
(10) Pub. No.: US 2016/0123982 A1
(43) Pub. Date: May 5, 2016

(54) METHODS FOR ASSAYING JAK2 ACTIVITY IN RED BLOOD CELLS AND USES THEREOF

(71) Applicants: INSERM (INSTITUT NATIONAL DE LA SANTÉ ET DE LA RECHERCHE MÉDICALE), Paris (FR); UNIVERSITÉ PARIS DIDEROT - PARIS 7, Paris (FR); INSTITUT NATIONAL DE LA TRANSFUSION SANGUINE (INTS), Paris (FR); UNIVERSITE DES ANTILLES ET DE LA GUYANE, Pointe-a-Pitre (FR)

(72) Inventors: Wassim EL NEMER, Paris (FR); Maria DE GRANDIS, Marseille (FR)

(21) Appl. No.: 14/765,631

(22) PCT Filed: Feb. 4, 2014
(86) PCT No.: PCT/EP2014/052153

§ 371 Date: Aug. 4, 2015

Related U.S. Application Data
(60) Provisional application No. 61/777,496, filed on Mar. 12, 2013.

(30) Foreign Application Priority Data
Feb. 4, 2013 (EP) 13305131.8

(51) Int. Cl. G01N 33/573

(52) U.S. Cl. CPC G01N 33/573; G01N 2333/912; G01N 2333/78; G01N 2800/7023; G01N 2800/52; G01N 2500/02; G01N 2500/10

(57) ABSTRACT

The present invention relates to methods for assaying JAK2 activity in a red blood cell. The present invention also relates to methods for diagnosing myeloproliferative neoplasm.

### FIELD OF THE INVENTION

The present invention relates to methods for assaying JAK2 activity in a red blood cell.

### BACKGROUND OF THE INVENTION

Polycythemia vera (PV) is a myeloproliferative neoplasm (MPN) characterized by the V617F activating mutation in the tyrosine kinase JAK2 (1-4). PV patients exhibit increased red cell mass, high platelet and leukocyte counts, with a high risk of thrombosis, considered as a major cause of mortality and morbidity in this disease (5).

In biomedical diagnostics, the V617F mutation is detected by a single nucleotide polymorphism genotyping assay performed with DNA extracted from granulocytes. When detected, the percentage of the mutated alleles is quantified by a PCR-based assay using the same DNA. In addition to its role in pathophysiology and diagnosis (6, 7), the JAK2V617F mutated form may be a prognostic marker because the percentage of circulating mutated alleles (% V617F) appears to be significantly higher in patients who develop vascular complications (8). The % V617F is considered as a good tool to monitor minimal residual disease and to evaluate treatment efficacy (9, 10).

Although the red cell lineage is primarily affected by the JAK2V617F mutation, the impact of this mutation on the behavior of circulating red blood cells (RBC) is poorly documented. The inventors investigated the interactions of RBCs with components of the vascular wall and showed that PV RBCs had an increased adhesion to endothelial cells mediated by endothelial laminin α5 chain and erythroid Lu/BCAM (11). Very recently, the inventors showed that this abnormal adhesion ensues the JAK2V617F-mediated Lu/BCAM activation through a Rap1/Akt signaling pathway (12). This was the first evidence that JAK2V617F was present and active in circulating PV RBCs.

The information about the presence, the activity and the percentage of JAK2V617F in circulating PV RBCs is still missing and incomplete. At the present time, in the absence of specific JAK2V617F antibodies, there is no technical approach and it is impossible to quantify this mutated form in PV RBCs. Quantifying a mutation by a PCR-based method in granulocytes does not reflect the mutated protein levels nor their activation in circulating blood cells in general, and in RBCs in particular. Therefore, the identification and development of an assay that measures the functional level of JAK2V617F activity in PV RBCs may be highly desirable.

### SUMMARY OF THE INVENTION

The present invention relates to a method for assaying JAK2 activity in a red blood cell comprising the steps consisting of i) bringing the red blood cell into contact with laminin ii) determining the ability of the red blood cell to adhere to laminin, and iii) concluding that JAK2 is activated when the red blood cell is able to adhere to laminin or concluding that JAK2 is not activated when the red blood cell is not able to adhere to laminin.

### DETAILED DESCRIPTION OF THE INVENTION

The role of the adhesion molecule Lu/BCAM as a potential biological marker of JAK2V617F activity in PV RBCs was investigated by the inventors in 17 JAK2V617F-positive PV patients. The inventors found a significant correlation between the number of adherent RBCs and the percentage of Lu/BCAM-positive RBCs. The inventors also demonstrated a strong correlation between RBC adhesion to laminin and the JAK2V617F percentage determined by PCR and represented as the allele burden. This correlation was reinforced in the group of 11 patients with similar percentages of Lu/BCAM-positive RBCs, demonstrating that JAK2V617F is the critical factor controlling the Lu/BCAM activation and adhesion level. The Lu/BCAM-mediated adhesion is determined as a biological effect of JAK2V617F activity in PV RBCs. The inventors' methods, based on a one-hour test, has a clear advantage over the current method used for determining the percentage of the mutated JAK2 allele in the total population of circulating nuclear blood cells and consisting of growing Endogenous Erythroid Colonies (EEC) by performing a two-week cell culture assay.

The present invention relates to a method for assaying JAK2 activity in a red blood cell comprising the steps consisting of i) bringing the red blood cell into contact with laminin ii) determining the ability of the red blood cell to adhere to laminin, and iii) concluding that JAK2 is activated when the red blood cell is able to adhere to laminin or concluding that JAK2 is not activated when the red blood cell is not able to adhere to laminin.

The term “JAK2” refers to a cytoplasmic tyrosine kinase that transduces signals triggered by multiple haemopoietic growth factors such as erythropoietin, in normal and neoplastic cells (1).

The term “JAK2 activity” refers to the kinase activity of JAK2. In particular JAK2 activity includes JAK2-mediated Lu/BCAM activation through a Rapl/Akt signaling pathway and activation of other proteins by phosphorylation such as ICAM-4/LW that interacts with integrin αvβ3 on the surface of endothelial cells.

The term “laminin” has its general meaning in the art and refers to the major component of the extracellular matrix. The term “laminin” refers to one of the major functional components of basement membranes, and are found underlying endothelium, and encasing pericytes and smooth muscle cells in the vessel wall. Laminins containing the α4 and α5 chains are the major isoforms found in the vessel wall, with the added contribution of laminin α2 in larger vessels.

Typically laminin is immobilized onto a solid support.

The term “solid support” refers to a material having a rigid or semi-rigid surface. Such materials will preferably take the form chips, plates, cuvettes, filters, titer plates, beads and the like, that have laminin to the surface of those supports. The supports are generally made of conventional materials, e.g., plastic polymers, cellulose, glass, ceramic, stainless steel alloy, and the like.

In some embodiments, the adhesion of the red blood cell to laminin may be determined by any well-known method in the art and typically involves adhesion assays such as described in the prior art (12). In particular, the adhesion ability is determined in flow condition. Typically an assay as described in the EXAMPLE is used for determining the adhesion of the red blood cell to laminin.

In a particular embodiment, the JAK2 activity may result from a JAK2 gain of function mutation.

Accordingly, the present invention also relates to a method for determining the presence of a JAK2 gain of function mutation in a red blood cell, comprising the steps consisting of i) bringing the red blood cell into contact with laminin ii) determining ability of the red blood cell to adhere to laminin, and iii) concluding that a JAK2 gain of function mutation is present in the red blood cell when the red blood cell is able to adhere to laminin or concluding that a JAK2 gain of function mutation is absent when the red blood cell is not able to adhere to laminin.

In this embodiment, the activation is not reached by adding to the red blood cell any agent capable of activating JAK2. The activation thus results only from the JAK2 gain of function mutation.

The term “JAK2 gain of function mutation” as used herein, refers to any mutation in JAK2 which has for consequence that the kinase activity is auto constitutive. The gain of function mutation can be a deletion, addition, or substitution of amino acids in the protein, which gives rise to the change in the function of the JAK2. In particular, the mutation is a somatic mutation. Typically a JAK2 gain of function mutation is the somatic JAK2V617F mutation described in James et al. (James C, Ugo V, Le Couedic J P, et al. A unique clonal JAK2 mutation leading to constitutive signalling causes polycythaemia vera. Nature. 2005;434:1144-1148). JAK2V617F is a point mutation (1849 G for T) in exon 14, causes the substitution of phenylalanine for valine at codon 617 in the JAK homology JH2 domain. Other JAK2 gain of function mutations include but are not limited to F537-K539delinsL, H538QK539L, K539L, N542-E543de1, E543-D544de1, R541-E543delinsK, I540-E543delinsMK and V617I.

The present invention also relates to a method for assaying the amount of constitutively active erythroid JAK2 (CAEJ) in a population of red blood cells due to a JAK2 gain of function mutation, comprising the steps consisting of i) bringing the population of red blood cells into contact with laminin and ii) determining the rate of red blood cells which adhere to laminin, wherein said rate is indicative of the amount of constitutively active erythroid JAK2 (CAEJ) in a population of red blood cells due to a JAK2 gain of function mutation.

The expression “amount of constitutively active erythroid JAK2” or “CAEJ amount” refers to the number of red blood cells comprising JAK2 with gain of function mutations; this number is shown by the inventors to reflect the allele burden of a JAK2 gain of function mutation. The expression “CAEJ amount” also reflects the percentage of the mutated JAK2 allele in the total population of circulating nucleated blood cells.

Typically, the rate is the number of adherent cells per surface unit (e.g. number of cells per mm²).

In some embodiments, the red blood cells may result from a blood sample derived from a patient suffering from a myeloproliferative neoplasm (MPN).

The term “myeloproliferative neoplasm” or “MPN” refers to myeloproliferative neoplasm such as revised in the World Health Organisation Classification D47.1. The term encompasses a number of entities characterized by uncontrolled marrow proliferation in the presence of intact cellular differentiation and includes polycythemia vera (PV), essential thrombocythemia (ET) and primary myelofibrosis (PMF).

The methods of the invention may find very different applications.

For example, the method for assaying the CAEJ amount may be particularly suitable for the prediction of a secondary event in patients suffering from a myeloproliferative neoplasm (MPN).

Accordingly, the present invention also relates to a method for determining the risk of a secondary event in a patient suffering from a myeloproliferative neoplasm comprising the steps consisting of i) determining the CAEJ amount in a sample of red blood cells obtained from the patient by performing the method as above described ii) comparing the CAEJ amount determined in step i) with a reference value and iii) concluding that the patient has a high risk of having a secondary event when the CAEJ amount determined at step i) is higher than the reference value, or concluding that the patient has a low risk of having a secondary event when the CAEJ amount determined at step i) is lower than the reference value.

Typically, the secondary event is selected from the group consisting of vascular complications, secondary myelofibrosis, pruritus and transformations into leukemia vascular complication.

The term “vascular complication” has its general meaning in the art and includes arterial and venous thrombosis.

In a particular embodiment, the reference value may be determined by carrying out a method comprising the steps of:

a) providing a collection of samples of red blood cells obtained from patients;

b) providing, for each blood sample provided at step a), information relating to the current clinical outcome (a secondary event or not);

c) providing a serial of arbitrary quantification values;

d) determining the CAEJ amount for each blood sample contained in the collection provided at step a);

e) classifying said samples of red blood cells in two groups for one specific arbitrary quantification value provided at step c), respectively: (i) a first group comprising samples of red blood cells that exhibit a quantification value for level that is lower than the said arbitrary quantification value contained in the said serial of quantification values; (ii) a second group comprising samples of red blood cells that exhibit a quantification value for said level that is higher than the said arbitrary quantification value contained in the said serial of quantification values; whereby two groups of samples of red blood cells are obtained for the said specific quantification value, wherein the samples of red blood cells of each group are separately enumerated;

f) calculating the statistical significance between (i) the quantification value obtained at step e) and (ii) the actual clinical outcome of the patients (i.e a secondary event) from which samples of red blood cells contained in the first and second groups defined at step f) derive;

g) reiterating steps f) and g) until every arbitrary quantification value provided at step d) is tested;

h) setting the said reference value as consisting of the arbitrary quantification value for which the highest statistical significance (most significant) has been calculated at step g).

For example the CAEJ amount has been assessed for 100 samples of red blood cells of 100 patients. The 100 samples are ranked according to CAEJ amount. Sample 1 has the highest CAEJ amount and sample 100 has the lowest CAEJ amount. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding patients, the p value between both subsets was calculated. The reference value is then selected such as the discrimination based on the criterion of the minimum p value is the strongest. In other terms, the CAEJ amount corresponding to the boundary between both subsets for which the p value is minimum is considered as the reference value. It should be noted that the reference value is not necessarily the median value of CAEJ amount. The setting of a single “cut-off” value thus allows discrimination between responder or non responder. Practically, high statistical significance values (e.g. low P values) are generally obtained for a range of successive arbitrary quantification values, and not only for a single arbitrary quantification value. Thus, in one alternative embodiment of the invention, instead of using a definite reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, on a hypothetical scale of 1 to 10, if the ideal cut-off value (the value with the highest statistical significance) is 5, a suitable (exemplary) range may be from 4-6. Therefore, a patient may be assessed by comparing values obtained by measuring the CAEJ amount, where values greater than 5 reveal that the patient has a high risk of having a secondary event and values less than 5 reveal that the patient has low risk of having a secondary event. In a another embodiment, a patient may be assessed by comparing values obtained by measuring the CAEJ amount and comparing the values on a scale, where values above the range of 4-6 indicate that the patient has a high risk of having a secondary event and values below the range of 4-6 indicate that the patient has a low risk of having a secondary event, with values falling within the range of 4-6 indicating an intermediate risk.

The methods of the invention may be also suitable for determining the presence or absence of the gain of function mutation in the erythroid lineage of a patient suffering from a myeloproliferative neoplasm. Determining the presence or absence of constitutively active JAK2 in the erythroid lineage has an impact on patient characterization within the three subpopulations of MPN patients: PV, ET or PMF. In addition, the presence of active JAK2 in the erythroid lineage of ET or PMF patients might give indications about their potential evolution into PV. The inventors' methods, based on a one-hour test, has a clear advantage over the current method used for the same purpose and consisting of growing Endogenous Erythroid Colonies (EEC) by performing a two-week cell culture assay.

The methods of the invention may be also suitable to define a subgroup of patients who are eligible for a treatment with a JAK2 inhibitor. Indeed patient with a high CAEJ amount may be administered with a JAK2 inhibitor.

JAK2 inhibitors are well known in the art (Tibes R, Bogenberger JM, Geyer HL, Mesa RA. JAK2 inhibitors in the treatment of myeloproliferative neoplasms. Expert Opin Investig Drugs. 2012 Dec;21(12):1755-74; Dymock BW, See CS. Inhibitors of JAK2 and JAK3: an update on the patent literature 2010 - 2012. Expert Opin Ther Pat. 2013 Apr;23(4):449-501) and include but are not limited to ruxolitinib (INCB018424), SAR302503 (TG101348),

Pacritinib (SB1518), CYT387, AZD-1480, BMS-911543, BMS-91153, NS-018, LY2784544, Lestaurtinib (CEP701), AT-9283, CP-690550, SB1578, R723, INCB16562, INCB20, CMP6, TG101209, SB1317 (TG02), XL-019, Baricitinib (LY3009104, INCB28050), XL-019 and compounds described in WO2012030944, WO2012030924, WO2012030914, WO2012030912, WO2012030910, WO2010099379, WO2012030894, WO2010002472, WO2011130146, WO2010038060, WO2010020810, WO2011028864, WO2010141796, WO2010071885, WO2011101806, S20100152181, WO2010010190, WO2010010189, WO2010051549, WO2011003065, WO2012022265, WO2012068440, WO2011028685, WO2010135621, WO2010039939, WO2012068450, WO2011103423, WO2011044481, WO2010085597, WO2010014453, WO2010011375, WO2010069966, WO2011097087, WO2011075334, WO2011045702, WO2010020905, WO2010039518, WO2010068710.

The methods of the invention are also particularly suitable for monitoring a treatment of patient suffering from a myeloproliferative neoplasm (e.g. with a JAK2 inhibitor). Typically a decrease in the CAEJ amount indicates that the treatment is effective in the patient.

The methods of the invention may be also particularly suitable for the in cellulo screening of JAK2 inhibitors. In particular, said method comprises the steps consisting of i) bringing a red blood cell harbouring a JAK2 gain of function mutation into contact with a candidate compound and ii) determining the ability of the red blood cell to adhere to laminin, wherein a decrease or an absence of adhesion indicate that the candidate compound is an effective in cellulo JAK2 inhibitor.

The invention will be further illustrated by the following figures and examples. However, these examples and figures should not be interpreted in any way as limiting the scope of the present invention.

### FIGURES

FIG. 1: PV RBC adhesion is weakly correlated with the percentage of Lu/BCAM-positive RBCs but not with Lu/BCAM MFI values.

RBC adhesion at 3 dyn/cm² as a function of the Lu/BCAM expression level on the surface of RBCs from 17 PV patients, represented as the percentage of circulating RBCs expressing Lu/BCAM (A) and Lu/BCAM MFI (B).

FIG. 2: PV RBC adhesion as a function of the JAK2V617F allele burden. RBC adhesion at 3 dyn/cm² as a function of the JAK2V617F percentage in all 17 patients (A) and in 11 patients with Lu/BCAM-positive RBCs percentage >65% (B). (C)

Typical microscopic images of RBC adhesion of PV patients with comparable percentages of Lu/BCAM-positive RBCs but different JAK2V617F percentages.

### EXAMPLE

Material & Methods

Patients and Blood Samples

The study was approved by the Internal Review Boards from participating institutions; blood samples were obtained with informed consent in accordance with the Declaration of Helsinki Seventeen PV patients were included in this study, 7 females and 10 males, with an average age of 67±12.01 years. All 17 patients were positive for JAK2V617F mutation and were seen as outpatients in the Cell Biology Department of the Hopital Saint-Louis, Paris, France. Patients were treated with phlebotomy, in addition to anticoagulants for 9 of them.

Adhesion Assays

Red blood cell adhesion to laminin 511/521 was measured under flow conditions using Vena8 Endothelial+™ biochips (internal channel dimensions: length 20 mm, width 0.8 mm, height 0.12 mm) and Mirus™ Nanopump (Cellix Ltd, Dublin, Ireland), as described (12). Laminin 511/521 from human placenta (Sigma-Aldrich) was immobilized on the internal surface of the biochips (1 μg/cm²) at 4° C. overnight. RBCs were suspended in Hanks balanced salt solution, without calcium chloride and magnesium sulfate (Sigma-Aldrich), supplemented with 0.4% BSA at hematocrit 0.5% and perfused through the biochip channels for 10 min at a shear stress of 0.2 dyn/cm². Five minutes washouts with the Hanks/0.4% BSA buffer were performed at 1, 2, 3, 4 and 5 dyn/cm². After each wash, adherent RBCs were counted in 11 representative areas along the centerline of each channel using the AxioObserver Z1 microscope and AxioVision 4 analysis software (Carl Zeiss, Le Pecq, France). Images of the same 11 areas were obtained throughout each experiment using the “Mark and Find” module of AxioVision analysis software.

Flow Cytometry

Cell surface expression of Lu/BCAM was analyzed using anti-Lu/BCAM F241 mouse mAb followed by a PE-conjugated secondary anti-mouse IgG antibody, and a BD FACScanto II flow cytometer (Becton-Dickinson, San Jose, CA) with FACSDiva software (v6.1.2) for acquisition and analysis.

Results

PV RBC Adhesion to Laminin is Variable and Weakly Correlated with Lu/BCAM Expression

The inventors showed that PV RBC adhesion to laminin was the consequence of

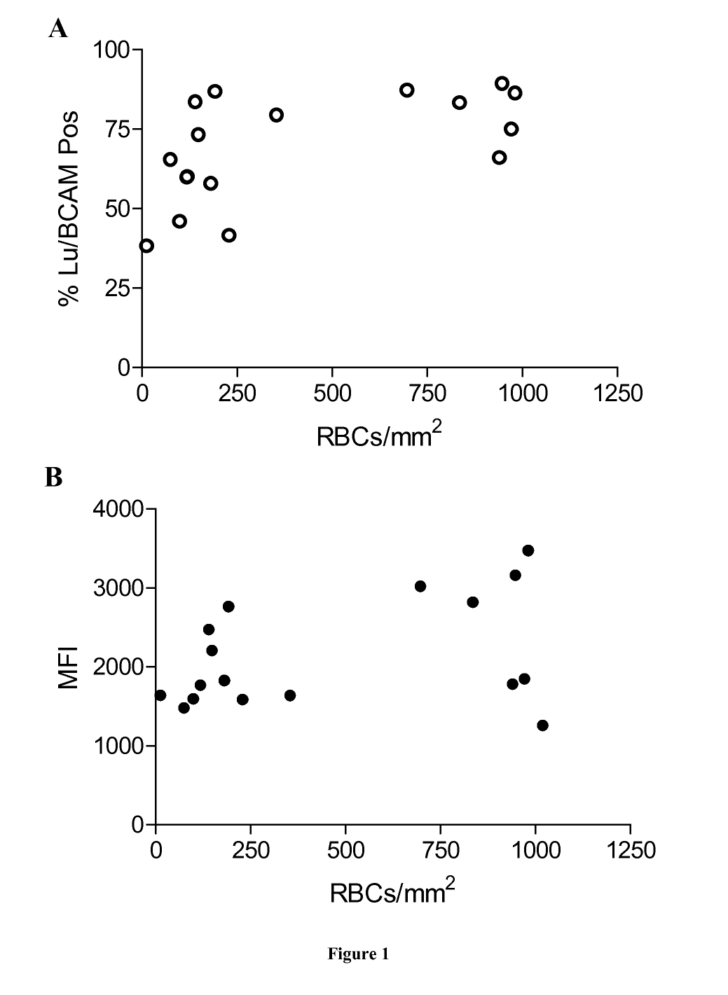

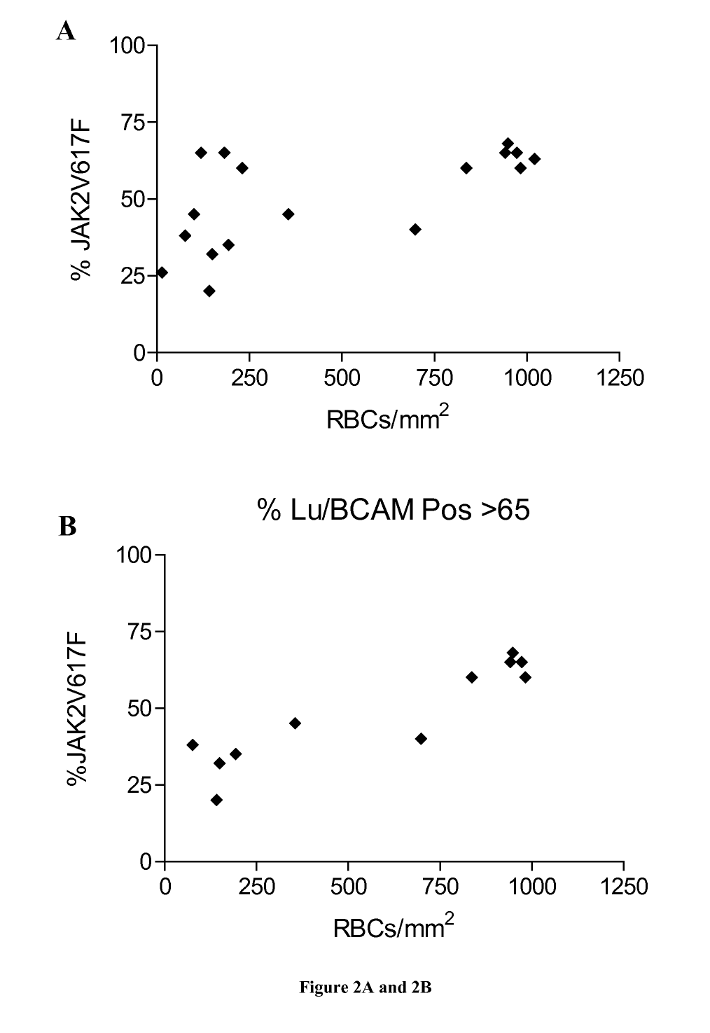

Lu/BCAM phosphorylation by a JAK2V617F/Rapl/Akt pathway (12). The inventors performed adhesion assays with blood samples from 17 JAK2V617F-positive PV patients and determined the number of RBCs adhering to laminin at 3 dyn/cm². The adhesion values were variable, ranging from 14 to 1021 RBC/mm² (Table 1). Because Lu/BCAM erythroid expression is heterogeneous, with a variable surface expression level among the population and a percentage of Lu/BCAM-negative RBCs within the same individual, the inventors asked whether PV RBC adhesion to laminin could be conditioned by Lu/BCAM expression pattern. Using a specific anti-Lu/BCAM antibody, the inventors performed flow cytometry analyses to characterize Lu/BCAM expression in all blood samples. The percentage of Lu/BCAM-positive RBCs ranged from 38 to 89.1, and the expression level, estimated by the mean fluorescence intensity (MFI), from 1249 to 3464 (Table 1). The number of adherent RBCs was plotted against the percentage of Lu/BCAM-positive RBCs or the MFI of each blood sample. The inventors found a weak correlation between the number of adherent RBCs and the percentage of Lu/BCAM-positive RBCs (r=0.4902 and P=0.0458 at 3 dyn/cm², FIG. 1A). No correlation was found between RBC adhesion and Lu/BCAM MFI values (r=0.3505 and P=0.1678 at 3 dyn/cm², FIG. 1B). These results highlight the central role of Lu/BCAM activation by JAK2V617F in the adhesion of PV RBCs to laminin. Indeed, RBCs from healthy volunteers express non-phosphorylated Lu/BCAM at their surface and do not adhere to laminin, indicating that the crucial factor driving the adhesion is not Lu/BCAM expression level but its activation state.

PV RBC Adhesion to Laminin Reflects the JAK2V617F Allele Burden

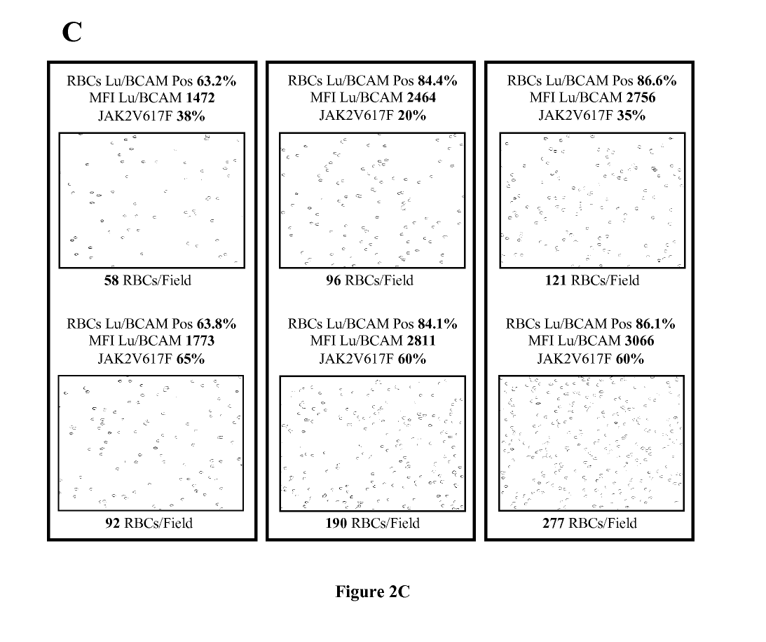

Because Lu/BCAM expression pattern failed to explain the variability of PV RBC adhesion, the inventors investigated the role of Lu/BCAM phosphorylation. Of note, a similar situation was described by our group in sickle cell disease, where increased RBC adhesion to laminin was correlated with Lu/BCAM phosphorylation (13). The inventors asked whether RBC adhesion was influenced by JAK2V617F expression level represented by the allele burden. The allele burden of JAK2V617F was determined by PCR at diagnosis for the 17 PV patients and ranged from 20% to 68% (Table 1). The previously determined adhesion values were plotted against the percentage of JAK2V617F. When all 17 patients were taken into account, a significant positive correlation was found between these two parameters (r=0.5715 and P=0.0166, FIG. 2A). In order to eliminate a potential disruptive parameter from the correlation, because of the wide range of Lu/BCAM-positive RBC percentage (38-89.1%)—shown above to influence cell adhesion—the inventors performed the same analysis in a smaller, more homogeneous group. The inventors selected those patients with a Lu/BCAM-positive percentage higher than 65%, within the 65-89.1% range (Table 1, patients 1 to 11). Statistical analysis showed a strong correlation between RBC adhesion and JAK2V617F percentage in this group (r=0.8493 and P=0.0009, FIG. 2B), indicating that, at comparable Lu/BCAM-positive percentages, higher JAK2V617F levels would drive higher RBC adhesion to laminin. In order to validate this result, adhesion assays were performed with RBCs from 3 pairs of PV patients showing comparable Lu/BCAM expression patterns but different JAK2V617F percentages. Patients with higher JAK2V617F showed higher RBC adhesion to laminin (FIG. 2C).

In the existing PCR-based test, the V617F mutation is detected in DNA samples extracted from patients' granulocytes. The mutation is thus detected in myelocytes but not in cells from the erythroid lineage. In addition, quantifying a mutation by a PCR-based method does not reflect the mutated protein levels. In this invention, the inventors revealed a biological marker that allows estimating the V617F mutational load at the protein level in PV RBCs. Inventor's assay does not quantify JAK2V617F mere expression but measures the functional level of this enzyme. In inventor's assay, the Lu/BCAM-mediated adhesion is determined as a direct biological effect of JAK2V617F activity in PV RBCs. This assay is interesting in other MPNs with partial JAK2V617F erythroid occurrence, such as Essential Thrombocythemia (ET), and primary myelofibrosis (PMF) as it discriminates between

JAK2V617F-positive and JAK2V617F-negative RBC samples and thus give an indication about the presence or absence of this mutation in the erythroid lineage of these patients.

TABLE 1
The list of the JAK2V617F-positive PV patients included in this
invention, numbered from 1 to 17, indicating their adhesion level
(RBCs/mm²), their relative values of Lu/BCAM expression
(% and MFI) and of JAK2V617F allele burden (% JAK2V617F).

| PV patient | RBC/mm² | % Lu/BCAM | MFI Lu/BCAM | % JAKV617F |
|---|---|---|---|---|
| 1 | 949 | 89.1 | 3153 | 68 |
| 2 | 699 | 87 | 3011 | 40 |
| 3 | 194 | 86.6 | 2756 | 35 |
| 4 | 983 | 86.1 | 3066 | 60 |
| 5 | 142 | 83.4 | 2464 | 20 |
| 6 | 837 | 83.1 | 2811 | 60 |
| 7 | 356 | 79.2 | 1628 | 45 |
| 8 | 973 | 74.8 | 1839 | 65 |
| 9 | 150 | 73 | 2197 | 32 |
| 10 | 942 | 65.8 | 1773 | 65 |
| 11 | 77 | 65.2 | 1472 | 38 |
| 12 | 1021 | 59.9 | 1249 | 63 |
| 13 | 120 | 59.6 | 1758 | 65 |
| 14 | 183 | 57.7 | 1819 | 65 |
| 15 | 101 | 45.8 | 1584 | 45 |
| 16 | 231 | 41.4 | 1577 | 60 |
| 17 | 14 | 38 | 1630 | 26 |

### REFERENCES

Throughout this application, various references describe the state of the art to which this invention pertains. The disclosures of these references are hereby incorporated by reference into the present disclosure.
1. Baxter E J, Scott L M, Campbell P J, et al. Acquired mutation of the tyrosine kinase JAK2 in human myeloproliferative disorders. Lancet. 2005;365 (9464): 1054-1061.
2. James C, Ugo V, Le Couedic J P, et al. A unique clonal JAK2 mutation leading to constitutive signalling causes polycythaemia vera. Nature. 2005;434(7037):1144-1148.
3. Kralovics R, Passamonti F, Buser A S, et al. A gain-of-function mutation of JAK2 in myeloproliferative disorders. N Engl J Med. 2005 ;352(17): 1779-1790.
4. Levine R L, Wadleigh M, Cools J, et al. Activating mutation in the tyrosine kinase JAK2 in polycythemia vera, essential thrombocythemia, and myeloid metaplasia with myelo fibrosis. Cancer Cell. 2005;7(4):387-397.
5. Schafer A I. Bleeding and thrombosis in the myeloproliferative disorders. Blood. 1984;64(1):1-12.
6. Tefferi A, Thiele J, Orazi A, et al. Proposals and rationale for revision of the World Health Organization diagnostic criteria for polycythemia vera, essential thrombocythemia, and primary myelo fibrosis: recommendations from an ad hoc international expert panel. Blood. 2007;110(4):1092-1097.
7. Vainchenker W, Constantinescu S N. A unique activating mutation in JAK2 (V617F) is at the origin of polycythemia vera and allows a new classification of myeloproliferative diseases. Hematology Am Soc Hematol Educ Program. 2005:195-200.
8. Vannucchi A M, Antonioli E, Guglielmelli P, et al. Prospective identification of high-risk polycythemia vera patients based on JAK2(V617F) allele burden. Leukemia. 2007;21(9):1952-1959.
9. Jones A V, Silver R T, Waghorn K, et al. Minimal molecular response in polycythemia vera patients treated with imatinib or interferon alpha. Blood. 2006;107(8):3339-3341.
10. Kiladjian J J, Cassinat B, Turlure P, et al. High molecular response rate of polycythemia vera patients treated with pegylated interferon alpha-2a. Blood. 2006;108(6):2037-2040.
11. Wautier M P, El Nemer W, Gane P, et al. Increased adhesion to endothelial cells of erythrocytes from patients with polycythemia vera is mediated by laminin alpha5 chain and Lu/BCAM. Blood. 2007;110(3):894-901.
12. De Grandis M, Cambot M, Wautier M P, et al. JAK2V617F activates Lu/BCAM-mediated red cell adhesion in polycythemia vera through an EpoR-independent Rapl/Akt pathway. Blood. 2012.
13. Bartolucci P, Chaar V, Picot J, et al. Decreased sickle red blood cell adhesion to laminin by hydroxyurea is associated with inhibition of Lu/BCAM protein phosphorylation. Blood. 2010;116(12):2152-2159.

1. A method for assaying JAK2 activity in a red blood cell comprising the steps of i) bringing the red blood cell into contact with laminin ii) determining the ability of the red blood cell to adhere to laminin, and iii) concluding that JAK2 is activated when the red blood cell is able to adhere to laminin or concluding that JAK2 is not activated when the red blood cell is not able to adhere to laminin.

2. A method for determining the presence of a JAK2 gain of function mutation in a red blood cell, comprising the steps of i) bringing the red blood cell into contact with laminin ii) determining ability of the red blood cell to adhere to laminin, and iii) concluding that a JAK2 gain of function mutation is present in the red blood cell when the red blood cell is able to adhere to laminin or concluding that a JAK2 gain of function mutation is absent when the red blood cell is not able to adhere to laminin.

3. A method for assaying the amount of constitutively active erythroid JAK2 in a population of red blood cells due to a JAK2 gain of function mutation, comprising the steps of i) bringing the population of red blood cells into contact with laminin and ii) determining the rate of red blood cells which adhere to laminin, wherein said rate is indicative of the amount of constitutively active erythroid JAK2 in a population of red blood cells due to a JAK2 gain of function mutation.

4. The method according to claim 3 wherein said red blood cells are from a blood sample derived from a patient suffering from a myeloproliferative neoplasm.

5. A method of predicting a secondary event in a patient suffering from a myeloproliferative neoplasm comprising the steps of i) determining an amount of constitutively active erythroid JAK2 (CAEJ) in a sample of red blood cells obtained from the patient by i) bringing the red blood cells into contact with laminin and ii) determining the rate of red blood cells which adhere to laminin, wherein said rate is indicative of the amount of CAEJ in the red blood cells iii) comparing the CAEJ amount determined in step ii) with a reference value and iv) concluding that the patient has a high risk of having a secondary event when the CAEJ amount determined at step ii) is higher than the reference value, or concluding that the patient has a low risk of having a secondary event when the CAEJ amount determined at step ii) is lower than the reference value.

6. The method according to claim 5 wherein said secondary event is selected from the group consisting of vascular complications, secondary myelofibrosis, pruritus and transformations into leukemia vascular complication.

7. The method according to claim 4, further comprising the step of concluding that a JAK2 gain of function mutation is present in the erythroid lineage of said patient if a JAK2 gain of function mutation is present in the red blood cells.

8. A method for identifying a patient who is eligible for a treatment with a JAK2 inhibitor, comprising the steps of i) determining an amount of constitutively active erythroid JAK2 (CAEJ) in a sample of red blood cells obtained from the patient by i) bringing the red blood cells into contact with laminin and ii) determining the rate of red blood cells which adhere to laminin, wherein said rate is indicative of the amount of CAEJ in the red blood cells iii) comparing the CAEJ amount determined in step ii) with a reference value and iv) concluding that the patient is eligible for a treatment with a JAK2 inhibitor when the CAEJ amount determined at step ii) is higher than the reference value, or concluding that the patient is not eligible for a treatment with a JAK2 inhibitor when the CAEJ amount determined at step ii) is lower than the reference value.

9. A method for monitoring efficacy of a JAK2 inhibitor treatment of a patient suffering from a myeloproliferative neoplasm, comprising
before and after administration of a JAK2 inhibitor to said patient
determining an amount of constitutively active erythroid JAK2 (CAEJ) in a sample of red blood cells obtained from the patient by
i) bringing the red blood cells into contact with laminin and
ii) determining the rate of red blood cells which adhere to laminin; and
if said rate is lower in said patient after administration of said JAK2 inhibitor, then concluding that said JAK2 inhibitor treatment is efficient, and if said rate is not lower in said patient after administration of said JAK2 inhibitor, then concluding that said JAK2 inhibitor treatment is not efficient.

10. The method according to claim 4, wherein said myeloproliferative neoplasm is polycythemia vera, essential thrombocythemia or primary myelofibrosis.

11. A method of in cellulo screening of JAK2 inhibitors for use in the treatment of myeloproliferative neoplasm wherein said method comprises the steps of i) bringing a red blood cell harbouring a JAK2 gain of function mutation into contact with a candidate compound and ii) determining the ability of the red blood cell to adhere to laminin, wherein a decrease or an absence of adhesion indicate that said candidate compound is an effective in cellulo JAK2 inhibitor.

12. The method according to claim 5, wherein said myeloproliferative neoplasm is polycythemia vera, essential thrombocythemia or primary myelofibrosis.

13. The method according to claim 6, wherein said myeloproliferative neoplasm is polycythemia vera, essential thrombocythemia or primary myelofibrosis.
